(19) United States
(12) Patent Application Publication
(10) Pub. No.: US 2026/0014309 A1
(43) Pub. Date: Jan. 15, 2026

(54) INTERMITTENT CATHETER BAG

(71) Applicant: ConvaTec Limited, Flintshire (GB)

(72) Inventors: Alice Young, Chester (GB); Gavin Roberts, Wiltshire (GB); Nicola Pilmer, Cheshire (GB)

(21) Appl. No.: 19/330,415

(22) Filed: Sep. 16, 2025

Related U.S. Application Data
(63) Continuation of application No. PCT/GB2024/050718, filed on Mar. 15, 2024.
(60) Provisional application No. 63/452,855, filed on Mar. 17, 2023.

(30) Foreign Application Priority Data
Apr. 3, 2023 (GB) 2304949.7

(51) Int. Cl. A61M 1/00; A61M 39/24

(52) U.S. Cl. CPC A61M 1/69; A61M 39/24; A61M 2202/0014; A61M 2202/0496

(57) ABSTRACT

A fluid collection bag connectable at a distal end of a catheter to receive fluid from the catheter. The fluid collection bag defined by at least two walls joined around a least a portion of their periphery. Where two apertures are arranged proximate to a first edge of the fluid collection bag and extending though the two walls. A line of weakness extends from the first edge between the two apertures.

### TECHNICAL FIELD OF THE INVENTION

The present invention relates to urinary catheters, in particular to fluid collection bags of urinary catheters.

### BACKGROUND TO THE INVENTION

A urinary catheter is an medical device comprising a hollow catheter tube designed for insertion into a user's bladder via the urethra to drain the bladder. Urinary catheters can be classified as either open catheters or closed catheters. In open systems the catheter can be drained via a funnel or similar component into a toilet or another receptacle that is separate to the catheter. In closed systems the catheter is drained directly into a fluid collection container connected directly to a drain on the catheter. For example, a closed catheter may be supplied with an integrated fluid collection bag into which fluid is drained for later disposal. The bag is typically sealed once filled to ensure no leaks until the user can empty the bag. To drain the bag a section may be opened, for example by tearing to provide a drainage channel.

Users can have problems in opening bag to drain it. This can happen because the bag must be robust enough to ensure its contents is not inadvertently released and so opening of the packaging may require careful manipulation as well as a large force. This can mean the user either cannot empty the bag as they cannot access it or that the package is not opened in a controlled manner. This can cause embarrassment for the user but also inconvenience as the difficulty in draining the bag may dissuade the user from using closed systems. These disadvantages can be particularly acute where the user has limited dexterity.

It is an object of embodiments of th present invention to at least partially overcome or alleviate the above problems and/or to provide an improved packaged catheter assembly.

### SUMMARY OF THE INVENTION

According to a broad aspect of the disclosure there is provided a fluid collection bag connectable to a catheter to receive fluid from the catheter. The fluid collection bag may be connectable at a distal end of the catheter. The fluid collection bag may be defined by at least two walls joined around at least a portion of their periphery. The fluid collection bag may comprise two apertures arranged proximate to a first edge of the fluid collection bag. The two apertures may extend through the two walls. A line of weakness may extend from the first edge between the two apertures.

According to a first aspect of the disclosure there is provided a fluid collection bag connectable at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures.

By providing a line of weakness between two apertures, the opening of the fluid collection bag for drainage is made easier. The line of weakness providing a preferential line of tearing requiring less force and the apertures providing a location where the user can hook their fingers and pull the two apertures apart instead of having to pinch the bag.

The two walls may be welded together. The welds may have a width of 5 mm. The welds may define welded regions. The apertures may be provided through the welds. The apertures may be provided in the welded regions.

The fluid collection bag may comprise a drainage edge. The fluid collection bag may comprise two lateral edges. The two lateral edges may be adjacent to the drainage edge. The fluid collection bag may be welded along the lateral edges. The two apertures may be arranged proximal to the drainage edge. The two apertures may be arranged along a lateral edge.

The fluid collection bag may have a longitudinal axis. The length of the fluid collection bag may be measured along the longitudinal axis. The length of features of the fluid collection bag may be measured along the longitudinal axis. The width of the fluid collection bag may be measured perpendicular to the longitudinal axis. The width of features of the fluid collection bag may be measured perpendicular to the longitudinal axis.

The fluid collection bag may comprise a cavity. The cavity may be defined by an inner surface of the two walls. The cavity may be defined by an inner edge of the joins. The cavity may be defined by an inner edge of the welds. The inner edge of the joins may be curved at corners.

The fluid collection bag may comprise a drain channel. The fluid collection bag may comprise a sealed drain channel. The drain channel may be integrally formed with the cavity. The drain channel may be elongate. The drain channel may extend from the cavity to the drainage edge. The drainage edge may be sealed. A port may be provided on the drainage edge. The drain channel may be in fluid communication with an exterior of the fluid collection bag via the port. The port may comprise a funnel. The port may comprise an attachment mechanism. The port may be configured to releasably attach to the distal end of the catheter. The drain channel may be defined by joins between the two walls. Two lateral edge joins may join the two walls along a portion of their lateral periphery. The drain channel may be defined by at least one drain join. The or each drain join may comprise a widened portion of the corresponding lateral edge. The drain channel may be defined by welded regions. The drain channel may have a width less than the cavity. The fluid collection bag may be configured to funnel liquid towards the drainage channel. The inner edge of the joins may be curved at an interface between the cavity and the drain channel. The inner edge of the joins may be sloped at an interface between the cavity and the drain channel. The drain channel may comprise a non-return valve. The non-return valve may be arranged in the drain channel. The non-return valve may be arranged in the drain channel between the drainage edge and a continuation of the line of weakness.

The non-return valve prevents fluid which has been collected in the bag from inadvertently draining out again, by having the non-return valve placed between the line of weakness and the inlet/drainage edge, when the upper part of the bag is torn, the valve in in the removed portion thereby allowing fluid to be drained from the bag.

Accordingly, in a preferred embodiment of the disclosure, there is provided a fluid collection bag connectable at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures, wherein the fluid collection bag comprises a drainage edge and two adjacent lateral edges, the two apertures being arranged proximal to the drainage edge, wherein the fluid collection bag further comprises a sealed drain channel, wherein a continuation of the line of weakness extends across the drain channel, and further comprising a non-return valve arranged in the drain channel between the drainage edge and the continuation of the line of weakness

The fluid collection bag may be joined along the lateral edges. The fluid collection bag may comprise lateral edge joins. The fluid collection bag may be welded along the lateral edges. The fluid collection bag may comprise lateral edge welds. The lateral edge welds may have a first width adjacent to the cavity and a second width adjacent to the drain. The first width may be less than the second width. The lateral edge joins may be wider adjacent to the drain. The fluid collection pouch may comprise drain welds. The drain welds may be adjacent to the drain. The two apertures may be provided in the drain weld. The or both drain welds may extend laterally inward from the lateral edge welds. The drain welds may comprise widened portions of the lateral edge welds.

The line of weakness may extend between a first lateral edge and a second lateral edge. The line of weakness may extend parallel to the drainage edge. The line of weakness may extend across (and preferably only across) the welded region. By providing the line of weakness only across the welded region, the integrity of the bag is not compromised, i.e. it does not increase the risk of rupture if the full bag is subjected to an external force. A continuation of the line of weakness may extend across the drain channel. That is, a line continuing along and beyond the line of weakness would extend across the drain channel. The line of weakness may comprise perforations. The line of weakness may comprise a line where the wall thickness is reduced.

The two apertures may be separated from the line of weakness by less than their width. The two apertures may be separated from the line of weakness by less than, 30 mm, preferably less than 20 mm, for example 10 mm The apertures may be arranged less than 100 mm apart, preferably less than 80 mm apart, more preferably less than 50 mm apart, for example less than 40 mm apart, or less than 30 mm apart, such as 28 mm apart. By providing the two apertures close together and close to the line of weakness the force required to tear the line of weakness is more directly applied, less force is used stretching the material between the aperture and the line of weakness.

The apertures may be finger holes. The apertures may be sized to accept a finger. The apertures may have the same shape. The apertures may have a different shape. One or both apertures may have a circular shape, a rectangular shape, a square shape, an oval shape, a stadium shape, a rounded rectangle shape. The apertures may be provided in a first drain join. The first drain join may be separated from the drainage edge by a portion of lateral edge join. The apertures may have a length of between 15 and 20 mm, for example 17 mm. The apertures may have a width of between 10 and 15 mm, for example 12.5 mm.

The fluid collection bag may comprise a tear stop aperture. The fluid collection bag may comprise two drain joins. The two drain joins may be on opposing lateral edges. The tear stop aperture may be provided in a second drain join. The tear stop aperture may be provided on the opposing side of the drain channel to the finger holes. A continuation of the line of weakness may intersect the tear stop aperture.

Accordingly, in a preferred embodiment of the disclosure, there is provided a fluid collection bag connectable at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures, the fluid collection bag further comprising a tear stop aperture.

In a further preferred embodiment of the disclosure, there is provided a fluid collection bag connectable at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures wherein the fluid collection bag further comprises a sealed drain channel, wherein a continuation of the line of weakness extends across the drain channel, wherein two lateral edge joins join the two walls along a portion of their periphery and the drain channel is defined by at least two drain joins, both or each drain join comprising a widened portion of the corresponding lateral edge join, wherein the two apertures are provided in a first drain join wherein the fluid collection bag comprises two drain joins on opposing lateral edges and a tear stop aperture is provided in a second drain join on the opposing lateral edge to the first drain join, wherein the fluid collection bag further comprises a pouring grip, wherein the pouring grip is provided proximal to a base edge on the same lateral edge as the tear stop aperture.

The drainage edge may be configured to remain attached to the fluid collection bag when the line of weakness is torn.

The first drain join may be narrower than the second drain join.

The fluid collection bag may comprise a pouring grip. The pouring grip may be an aperture. The pouring grip may be provided proximal to a base edge. The pouring grip aperture may be provided proximate to a corner between a base edge and a lateral edge. The pouring grip may be provided on the same lateral edge as the tear stop aperture.

It is particularly advantageous that a pour grip be provided on the same lateral edge as the tear stop aperture. Once the bag is torn the drainage edge will be connected to the lateral edge on which the tear stop was provided, by having the pour grip on the same edge the pouring motion will direct this edge to be uppermost, ensuring the drainage edge is not caught in fluid whilst drained.

Accordingly, in a preferred embodiment of the disclosure there is provided a fluid collection bag connectable at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures, the fluid collection bag further comprising a tear stop aperture and a pouring grip, wherein the pouring grip are provided on a same lateral edge of the fluid collection bag.

In a further preferred embodiment of the disclosure, there is provided a fluid collection bag connectable at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures wherein the fluid collection bag further comprises a scaled drain channel, wherein a continuation of the line of weakness extends across the drain channel, wherein two lateral edge joins join the two walls along a portion of their periphery and the drain channel is defined by at least two drain joins, both or each drain join comprising a widened portion of the corresponding lateral edge join, wherein the two apertures are provided in a first drain join wherein the fluid collection bag comprises two drain joins on opposing lateral edges and a tear stop aperture is provided in a second drain join on the opposing lateral edge to the first drain join, wherein the fluid collection bag further comprises a pouring grip, wherein the pouring grip is provided proximal to a base edge on the same lateral edge as the tear stop aperture.

The fluid collection bag may be any suitable shape or size, for example rectangular, circular, elliptical, cuboid, spherical, etc. The two walls may be a front panel and a rear panel. The fluid collection bag may comprise a peripheral bond joining the periphery of the front panel and rear panel to form the fluid collection bag. The peripheral bond may define a base, two lateral edges and an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag.

The bag may have a width between the left lateral edge and right lateral edge of at least 10, 12, 15 or 20 cm. The bag may have a width no more than 25, 20, or 15 cm. Preferably, the width is between 10 and 20 cm, most preferably between 12 and 15 cm. The base may define a bottom of the bag. The upper edge may define a top of the bag. The length/height of the bag from the base to the upper edge may be at least 15, 20, 25, 30, 35 or 40 cm. The height of the bag may be no more than 45, 40, 35, 30, 25, or 20 cm. The height may be between 20 and 50 cm, 30 and 40 cm, or most preferably 32 cm. The bag may be configured to hold at least 500, 700, 1000, or 1200 ml of fluid, most preferably at least 700 ml of fluid.

The fluid collection bag may comprise a flexible plastics material. For example, polypropylene (PP), polyethylene terephthalate (PET), low density polyethylene (LDPE), metalized polyester (MET PET) orientated polypropylene (OPP) or polyvinyl chloride (PVC). At least part of the fluid collection bag may be transparent or translucent. The front panel of the fluid collection bag may be transparent or translucent. Thus, the fluid collection bag can conform to the shape of the catheter assembly, is convenient to carry and also provides a visual reference to the contents of the bag before, during and after use which makes it easier to use.

The fluid collection bag may be connected to a catheter. The fluid collection bag may be connected at a distal end to a catheter.

Accordingly, according to a second aspect of the disclosure there is provided a closed urinary catheter assembly comprising a fluid collection bag connected at a distal end of a catheter to receive fluid from the catheter, the fluid collection bag defined by at least two walls joined around at least a portion of their periphery; the fluid collection bag comprising two apertures arranged proximate to a first edge of the fluid collection bag and extending through the two walls; wherein a line of weakness extends from the first edge between the two apertures.

The fluid collection bag of the second aspect may be the fluid collection bag of the first aspect, including any optional features thereof.

According to a third aspect of the disclosure there is provided a method of draining a fluid collection bag connectable at a distal end of a catheter to receive fluid from the catheter, the fluid collection bag defined by at least two walls joined around a least a portion of their periphery; two apertures arranged proximate to a first edge of the fluid collection bag and extending though the two walls; wherein a line of weakness extends from the first edge between the two apertures, the method comprising: inserting at least a portion of a finger into each of the two apertures, pulling the two apertures apart to initiate a tear in the line of weakness, continuing the tear into a cavity of the fluid collection bag to create an opening; draining fluid from the fluid collection bag via the opening.

The fluid collection bag of the third aspect may be the fluid collection bag of the first or second aspects, including any optional features thereof.

The fluid collection bag may further comprise a drain. The method may further comprise continuing the tear across the drain. The method may further comprise continuing the tear to a tear stop aperture. The method may further comprise retaining the drainage edge of the fluid collection bag by an edge of the tear stop aperture.

### DETAILED DESCRIPTION OF THE INVENTION

In order that the invention may be more clearly understood one or more embodiments thereof will now be described, by way of example only, with reference to the accompanying drawings, of which:

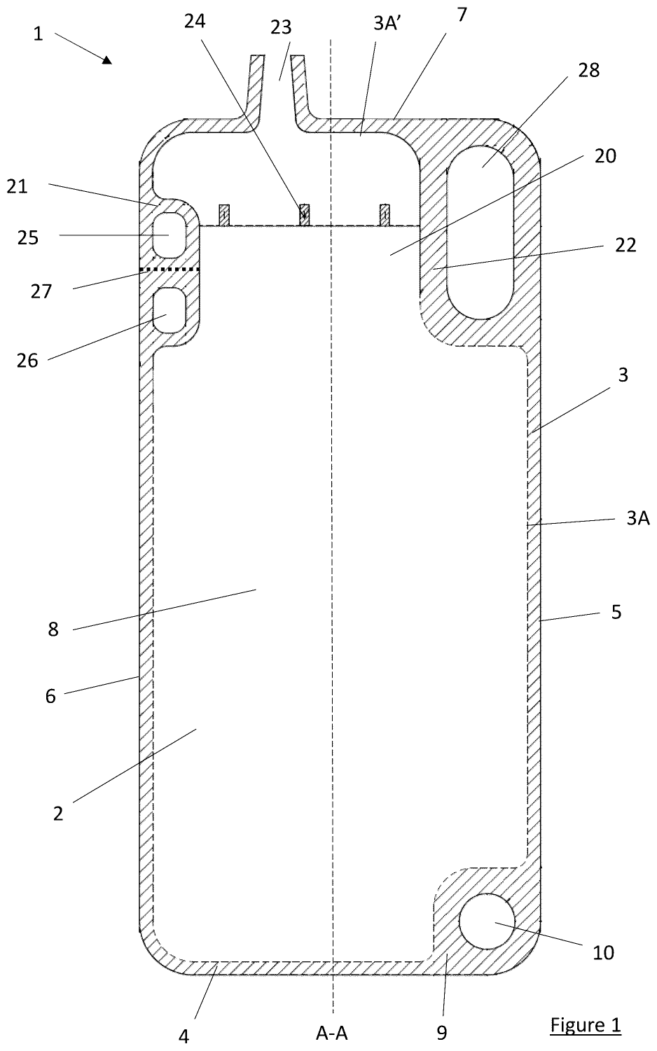

FIG. 1 shows a front view of a fluid collection bag according to an aspect of the present invention configured for collection;

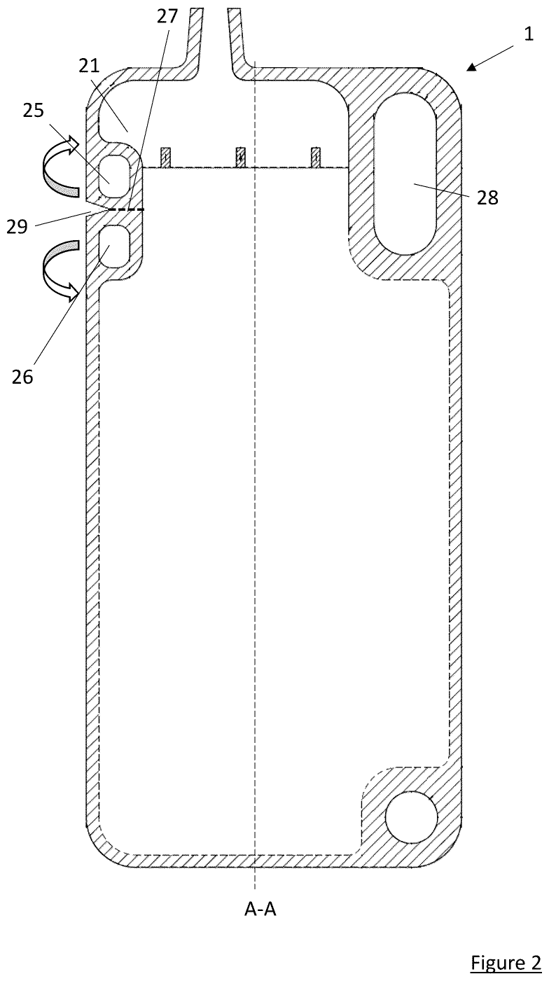

FIG. 2 shows a front view of the fluid collection bag of FIG. 1 where a tear is initiated;

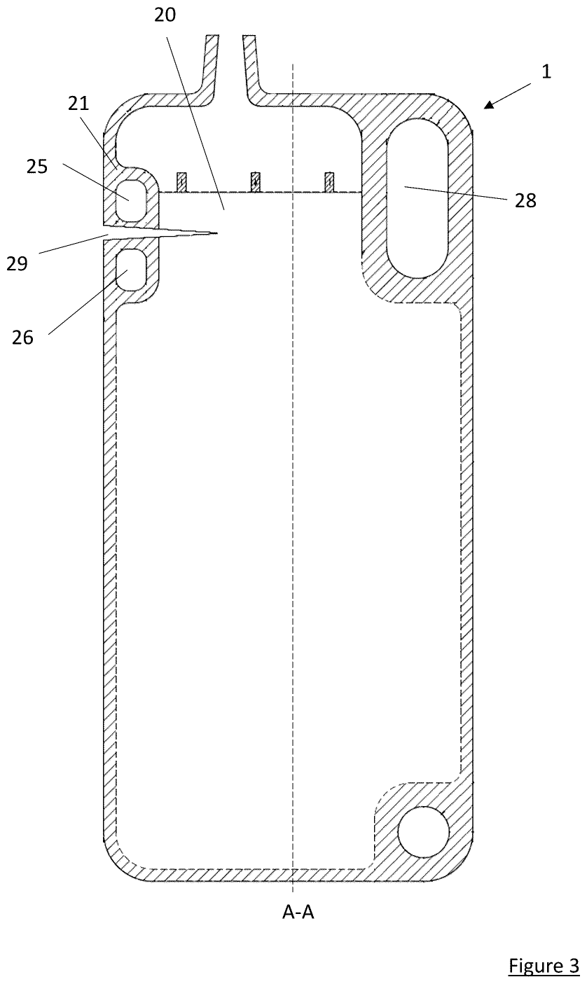

FIG. 3 shows a front view of the fluid collection bag of FIG. 1 where a tear continues into a drain;

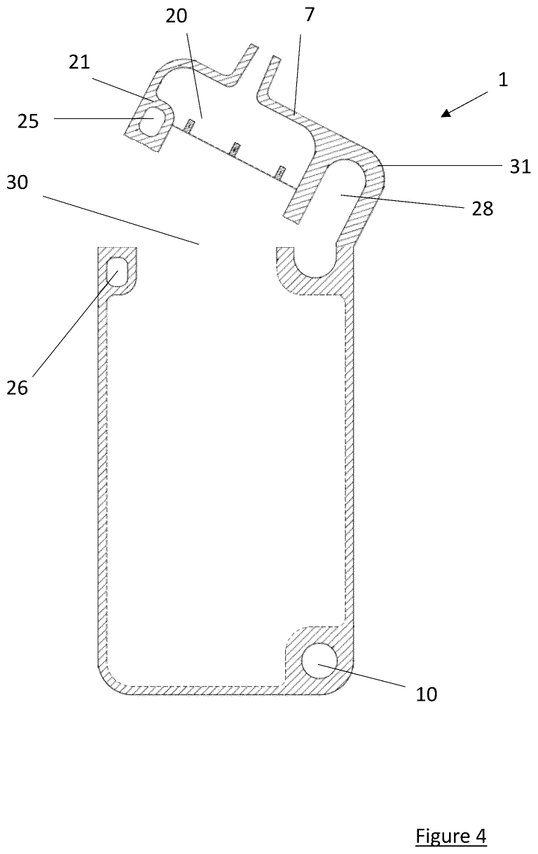

FIG. 4 shows a front view of the fluid collection bag of FIG. 1 where the drainage edge is removed; and

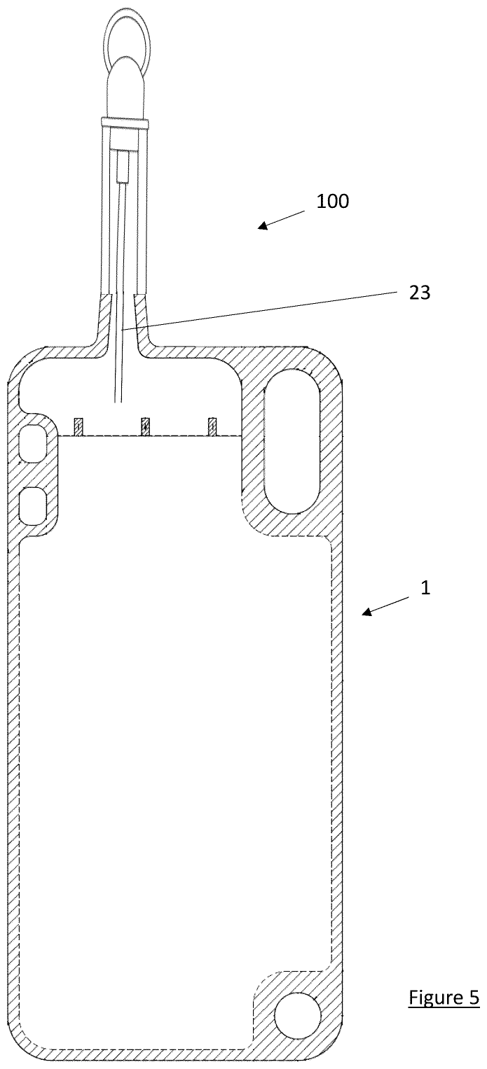

FIG. 5 a perspective view of a closed urinary catheter assembly including a fluid collection bag according to an aspect of the present invention.

With reference to FIG. 1 a fluid collection bag 1 is described. The fluid collection bag comprises a front panel 2, a rear panel (not shown) of identical shape and size to the front panel 2, and a peripheral bond 3 joining the periphery of the panels 2 to form the bag 1.

In this embodiment, the peripheral bond 3 defines a base 4, a right lateral edge 5, a left lateral edge 6 and an upper edge 7 of the bag 1. To aid in the description the fluid collection bag 1 can be considered to have a longitudinal axis A-A extending along a centre of the bag 1 between the base 4 and upper edge 7. An inner edge of the peripheral bond 3A and inner surfaces of the front 2 and rear panels define a cavity 8. To aid in the flow of fluid within the bag, in particular during draining, the inner edge of the peripheral bond 3A may be curved at the corners.

The peripheral bond thus defines a bag 1 that is generally rectangular and suitably has a width between the lateral edges 5,6 of between 10 and 20 cm, and most preferably between 12 and 17 cm between 120 and 170 mm, for example about 150 mm, and a height from the base 4 to the upper edge 7 of between 25 and 40 cm, 30 and 35 cm, or most preferably 30 and 33 cm, for example about 32 cm. The bag is thus configured to hold at least 700 ml, preferably at least 1000 ml of fluid released from the user through the catheter safely within the cavity 8. The base 4 defines the bottom of the bag 1 in use, and the upper edge 7 the top. However, in other embodiments alternative shapes and sizes could be conceived, such as elliptical or circular panels forming correspondingly shaped bags, or even a tubular bag or bag with more than two panels, e.g. a prism or other polygon. To improve the form of the bag 4, the corners between the edges 3,4,5,6 may be rounded.

In this embodiment, the panels comprise a flexible plastics material, for example polypropylene (PP), polyethylene terephthalate (PET), low density polyethylene (LDPE), metalized polyester (MET PET), orientated polypropylene (OPP) or polyvinyl chloride (PVC). The panels are preferably transparent, but may be translucent. In other embodiments the panels may be opaque. In particular, the front panel 2 may be transparent/translucent, but the rear panel opaque.

To define different portions of the bag, on the lateral edges 5,6 the peripheral bond 3 extends in from the edge by varying amounts. Extending from the top edge 7 and approximately 25% of the length of the bag 1 the width of the peripheral bond 3 on the right lateral edge 5 is increased such that the bonded width is approximately 9 times the width of the lateral peripheral bond 3. The peripheral bond 3 of the left lateral edge is also wider proximate to the top edge 7, though not extending to the top edge 7 so as to avoid a large area of weld which may crease during the welding step and thus be unsightly. These widened portions of the peripheral bond 3 define a drain 20 (details of which will be provided below) and thus the peripheral bond 3 adjacent to the drain 20 may be referred to as a left drain bond 21 and a right drain bond 22. The lower edges of the left 21 and right 22 drain bonds are equal distance from the top edge 7 and define an upper extent of the cavity 8.

Where the base 4 and right lateral edge 5 meet there may be provided a generally square shaped base bonded region 9. Arranged within the base bonded region 9 there is provided a pouring aperture 10 which extends through the front 2 and rear panels, the pouring aperture 10 provides a point that which the user can grip the bag 1 when draining the bag 1. It is preferable that the pouring aperture 9 is sized to allow a user's finger to at least partially be inserted through, accordingly the aperture may be any suitable shape, for example a circle, an oval, an ellipse, a polygon, a triangle, a square, a rounded square, a stadium etc. As will be appreciated by those skilled in the art, the pouring aperture 9 may alternatively be provided where the base 4 and left lateral edge 6 meet. It will also be appreciated that an aperture need not be provided, the outer surface of the panels may be provided with a textured surface in the base bonded region to improve grip.

As noted above the drain 20 is provided proximal to the upper edge 7, it is integrally formed with the cavity 8, it has an elongate shape and extends from an inner edge 3A′ of the peripheral bond on the top edge 7. The drain 20 may be in fluid communication with an exterior of the bag 1 via an inlet 23. The inlet 23 is provided by an elongate projection of the front 2 and rear walls welded along their lateral edges. Alternatively, the inlet 23 may be provided between the front 2 and rear panels through the peripheral bond 3. The inlet 23 may comprise a port adapted to connect to a catheter, alternatively the inlet 23 may be connected to a distal end of a catheter 100 as shown in FIG. 5. Where the fluid collection bag is connected to the distal end of a catheter it will be recognised that this is a “closed” catheter assembly.

Arranged in the drain 20 and extending across the width thereof there is provided a non-return valve 24. The non-return valve prevents fluid collected in the cavity 8 from escaping via the inlet 23.

The left drain bond 21 and the right drain bond 22 may have an equal width such that the drain 20 is arranged centrally with respect to the longitudinal axis A-A, or as in the present embodiment the drain 20 may be laterally offset with one of the drain bonds 21,22 being wider than other (in this case the right drain bond 22 is wider than the left drain bond 21).

The narrower left drain bond 21 is provided with two apertures extending through the front 2 and rear panels. The apertures are displaced along the longitudinal axis from one another, the aperture nearest the upper edge 7 is an upper finger hole 25 and the other aperture is a lower finger hole 26. The upper and lower finger holes 25,26 are sized to allow a user's finger to at least partially be inserted through, accordingly the aperture may be any suitable shape, for example a circle, an oval, an ellipse, a polygon, a triangle, a square, a rounded square, a stadium etc. In the present embodiment they are stadia with a length of 17 mm and a width on 12.5 mm.

Arranged between the finger holes 25,26 is a line of weakness 27. The line of weakness 27 extends perpendicularly from the left lateral edge 6 across the left drain bond 21 to the edge of the drain 20. The line of weakness 27 is configured to allow preferential tearing along its length, in this embodiment the line of weakness comprises a line of perforations, in alternative embodiments the preferential tearing may be achieved by other means such as where the panels are thinner along the line of weakness 27. The line of weakness 27 is positioned such that the non-return valve 24 is arranged between the line of weakness 27 and the upper edge 7 of the bag 1.

The right drain bond 22 is provided with a tear stop aperture 28 extending between the front 2 and rear panels. In this embodiment the tear stop aperture 28 has a stadium shape with the long axis extending parallel to the longitudinal axis A-A of the bag 1. The tear stop aperture 28 intersects a continuation of the line of weakness 27.

With reference to FIGS. 2-5 the method of draining the fluid collection bag 1 is described. Once the user has filled the bag 1 the inlet 23 may be sealed allowing the fluid collection bag 1 to be stored for later draining.

When the user wishes to drain the bag 1, they may grip the bag 1 with both hands, optionally inserting a finger from one hand at least partially into the upper finger hole 25 and a finger from the other hand at least partially into the lower finger hole 26. Then as shown in FIG. 2 the user pulls the finger holes 25,26 in opposing directions, one in the direction the front panel faces and one in the direction the rear panel faces. Alternatively, the two finger holes 25,26 may be separated in the direction of the longitudinal axis. This action initiates a tearing action along the line of weakness 27 creating a tear 29 in the left lateral edge 6. Providing finger holes 25,26 through which at least a part of the user's finger can be inserted improves the ease with which the user can initiate the tear. This is particularly advantageous for users with reduces dexterity who may struggle to grip the bag 1 in a pinch hold, but can hook a finger into each of the finger holes 25,26.

After the tear 29 is initiated, the user continues to pull the two finger holes 25,26 and the tear 29 continues along the line of weakness 27 and into the drain 20 as shown in FIG. 3. As can be seen in FIG. 3, the tear is directed by the line of weakness to cross the drain such that the non-return valve 24 is separated from the cavity 8, this allows the fluid to be drained without the valve 24 stopping the flow.

The tear 29 continues until it reaches the tear stop aperture 28 as shown in FIG. 4, the upper edge 7 is freed from the bag 1 and the drain 20 is now open to an environment exterior to the bag 1 via a drain aperture 30. A portion of the peripheral seal 3 between the tear stop aperture 28 and the upper and right later edges form a retention strip 31 retaining the upper edge 7 of the bag to the remainder of the bag 1. The user may now grip the bag by the tear stop aperture 28 and the pouring aperture 9 and tip the bag 1 such that the left lateral edge 6 is lowermost, the stored fluid thereby flows out of the drain aperture 30, once emptied the bag 1 may be disposed of.

The one or more embodiments are described above by way of example only. Many variations are possible without departing from the scope of protection afforded by the appended claims.

1. A fluid collection bag connectable at a distal end of a catheter to receive fluid from the catheter, the fluid collection bag defined by at least two walls joined around a least a portion of their periphery; two apertures arranged proximate to a first edge of the fluid collection bag and extending though the two walls; wherein a line of weakness extends from the first edge between the two apertures.

2. A fluid collection bag according to claim 1 wherein the two walls are welded together and the apertures are provided through the weld.

3. A fluid collection bag according to claim 1 or 2 wherein the fluid collection bag comprises a drainage edge and two adjacent lateral edges, the two apertures being arranged proximal to the drainage edge.

4. A fluid collection bag according to claim 3 wherein the line of weakness extends parallel to the drainage edge.

5. A fluid collection bag according to claim 3 or 4 wherein the two apertures are arranged along one of the lateral edges.

6. A fluid collection bag according to any preceding claim wherein the two apertures are arranged less than 5 cm apart.

7. A fluid collection bag according to any preceding claim, wherein the fluid collection bag further comprises a sealed drain channel, wherein a continuation of the line of weakness extends across the drain channel.

8. A fluid collection bag according to claim 7 wherein two lateral edge joins join the two walls along a portion of their periphery and the drain channel is defined by a at least one drain join, the or each drain join comprising a widened portion of the corresponding lateral edge join.

9. A fluid collection bag according to claim 8 wherein the two apertures are provided in a first drain join.

10. A fluid collection bag according to any preceding claim, further comprising a tear stop aperture.

11. A fluid collection bag according to claim 9 wherein the fluid collection bag comprises two drain joins on opposing lateral edges and a tear stop aperture is provided in a second drain join on the opposing lateral edge to the first drain join.

12. A fluid collection bag according to claim 11 wherein the continuation of the line of weakness intersects the tear stop aperture.

13. A fluid collection bag according to any preceding claim wherein the line of weakness comprises perforations.

14. A fluid collection bag according to claim 9 or claims 11 to 13 when dependent upon claim 9 wherein the line of weakness extends across the first drain join.

15. A fluid collection bag according to claim 11 or claims 12 to 14 when dependent upon claim 11 wherein there first drain join is narrower than the second drain join.

16. A fluid collection bag according to claim 11 or any of claims 12 to 15 when dependent upon claim 10 wherein the fluid collection bag further comprises a pouring grip, wherein the pouring grip is provided proximal to a base edge on the same lateral edge as the tear stop aperture.

17. A fluid collection bag according to claim 7 when dependent upon claim 3 or any claim directly or indirectly dependent upon claim 7 when dependent upon claim 3 further comprising a non-return valve arranged in the drain channel between the drainage edge and the continuation of the line of weakness.

18. A fluid collection bag according to any preceding claim wherein an inner edge of the peripheral join is rounded at corners.

19. A catheter assembly comprising a fluid collection bag connected at a distal end of a catheter to receive fluid from the catheter, the fluid collection bag defined by at least two walls joined around a least a portion of their periphery; two apertures arranged proximate to a first edge of the fluid collection bag and extending though the two walls; wherein a line of weakness extends from the first edge between the two apertures.

20. A catheter assembly according to claim 19 wherein the fluid collection bag is the fluid collection bag according to any of claims 1 to 18.

21. A method of draining a fluid collection bag connectable at a distal end of a catheter to receive fluid from the catheter, the fluid collection bag defined by at least two walls joined around a least a portion of their periphery; two apertures arranged proximate to a first edge of the fluid collection bag and extending though the two walls; wherein a line of weakness extends from the first edge between the two apertures, the method comprising:
inserting at least a portion of a finger into each of the two apertures,
pulling the two apertures apart to initiate a tear in the line of weakness,
continuing the tear into a cavity of the fluid collection bag to create an opening;
draining fluid from the fluid collection bag via the opening.

22. A method of draining a fluid collection bag according to claim 21 wherein the fluid collection bag is a fluid collection bag according to any of claims 1 to 18, or the fluid collection bag of a catheter assembly according to claim 19 or 20.

23. A method of draining a fluid collection bag according to claim 21 or 22 wherein the fluid collection bag further comprises a drain, wherein the method further comprises continuing the tear across the drain.

24. A method of draining a fluid collection bag according to any of claims 21 to 23 when dependent upon claim 10 or 11, wherein the tear continues to the tear stop aperture.

25. A method of draining a fluid collection bag according to claim 24 wherein the drainage edge of the fluid collection bag is retained by an edge of the tear stop aperture.
